(19) United States
(12) Patent Application Publication
(10) Pub. No.: US 2003/0109529 A1
(43) Pub. Date: Jun. 12, 2003

(54) Use of s-triazines for treating Apicomplexan parasitic infections

(75) Inventors: Miles P. Hacker, Rockford, MI (US); Greg Manske, St. Albans, VT (US); Lori L. Shatney-Leach, Montpelier, VT (US)

Correspondence Address: MORGAN LEWIS & BOCKIUS LLP, 1111 PENNSYLVANIA AVENUE NW, WASHINGTON, DC, 20004, US

(73) Assignee: Biomes, Inc., Amherst, MA

(21) Appl. No.: 10/261,622

(22) Filed: Oct. 2, 2002

Related U.S. Application Data
(62) Division of application No. 09/481,509, filed on Jan. 12, 2000, now abandoned.
(60) Provisional application No. 60/115,861, filed on Jan. 13, 1999.
(60) Provisional application No. 60/121,474, filed on Feb. 24, 1999.
(60) Provisional application No. 60/120,758, filed on Feb. 19, 1999.

(51) Int. Cl. A61K 31/53

(52) U.S. Cl. 514/241; 514/245

(57) ABSTRACT

This invention relates to novel compositions and methods of treating humans and animals infected by Apicomplexan parasites. More specifically, the present invention relates to treating an Apicomplexan infection by administering s-triazines, such as atrazine, to an infected human or animal. The present invention also relates to pharmaceutical compositions containing therapeutically effective amounts of s-triazines, such as atrazine, useful in treating parasitic infections.

### CROSS REFERENCES TO RELATED APPLICATIONS

This application claims priority to U.S. Provisional Patent Application No. 60/115,861, filed on Jan. 13, 1999; U.S. Provisional Patent Application No. 60/120,758, filed on Feb. 19, 1999; and U.S. Provisional Patent Application No. 60/121,474, filed on Feb. 24, 1999, each of which is incorporated in its entirety by reference.

### FIELD OF THE INVENTION

The present invention pertains, in general, to novel compositions and associated methods of treating humans and animals infected by Apicomplexan parasites. In particular, the present invention pertains to treating an Apicomplexan infection by administering a s-triazine, such as atrazine, to an infected human or animal.

### BACKGROUND OF THE INVENTION

All publications and patent applications herein are incorporated by reference to the same extent as if each individual publication or patent application was specifically and individually indicated to be incorporated by reference.

Apicomplexans.

Apicomplexans are microorganisms which contain a plastid-like organelle. Phylogenetic analyses indicate that Apicomplexans may have acquired the plastids by secondary endosymbiosis, e.g., from a green algae (Fiehera and Roos, 1997, Nature 390(6658):407-409; Kohler et al., 1997, Science 275(5305):1485-1489).

Parasites of the phylum Apicomplexa include many important human and veterinary pathogens such as Plasmodium sp (protozoans that are parasites of the red blood cells of vertebrates and include the causative agents of malaria), Toxoplasma sp (an opportunistic infection associated with AIDS and congenital neurologic birth defects), Neospora sp (an economically significant disease of poultry and cattle), Cryptosporidium sp (parasitic coccidian protozoans that infect the epithelial cells of the gastrointestinal tract in vertebrates and flourish in humans under conditions of intense immunosuppression), Hematodinium sp, Hemogregarines sp, Babesia sp (sporozoans that infect the red blood cells of humans and of animals such as dogs, cattle and sheep), Eimeria sp (coccidial protozoa that infects red blood cells, especially in young domesticated mammals and birds), and Theileria sp. For a more complete list of the parasitic species within the Apicomplexa phylum, see the Taxonomy Browser website of The National Center for Biotechnology Information (www.ncbi.nlm.nih.gov/htbin-post/Taxonomy); Ash and Orihel, 1997, Atlas of Human Parasitology, Fourth Edition; Levine and Ivens, 1986, The Coccidian Parasites (Protozoa, Apicomplexa of Artiodactyla), University of Illinois Press; Canning et al., 1986, The Microsporidia of Vertebrates, Academic Press; and Markell et al., 1992, Medical Parasitology, W. B. Saunders Co.

Apicomplexans cause substantial morbidity, mortality and economic losses to humans and animals, and new medicines to treat them are needed urgently (Roberts et al., 1998, Nature 393(6687):801-805).

Malaria.

Malaria remains one of the world's most devastating human infections, with 300 to 500 million clinical cases and nearly 3 million deaths per year (Tracy and Webster, 1996, Drugs Used in the Chemotherapy of Protozoal Infections: Malaria, Chapter 40:965-985, In Goodman & Gilman's The Pharmacological Basis of Therapeutics, Ninth Edition, McGraw-Hill). Malaria may reach 70 to 80% or more among children in hyperendemic areas during the transmission season. Thus, its impact on the health of the developing world is enormous.

Malaria is an infectious disease characterized by cycles of chills, fever and sweating associated with the synchronous lysis of red blood cells parasitized by a protozoan of the genus Plasmodium. Four plasmodia produce malaria in humans: Plasmodium falciparum, P. vivax, P. ovale and P. malariae. The disease is transmitted by the bite of an infected female anopheles mosquito. For a more complete description of the life cycle, epidemiology, pathology, diagnosis and clinical manifestations of malaria parasites, see Krogstad, 1996, Malaria, Chapter 374:1893-1896, In Cecil Textbook of Medicine, Bennett and Plum; and Berkow et al. (eds.), 1992, The Merck Manual, Sixteenth Edition, Chapter 15:229-232.

As the world waits for an effective anti-malarial vaccine to be developed, we must continue to rely on drug treatments to combat the disease. A major problem of current drug therapies is the growing number of malarial strains resistant to established chemotherapeutic agents, including strains resistant to isoquinolines and antifolate drugs. Of particular importance is the increasing prevalence of P. falciparum resistance to the drug chloroquine. P. falciparum is now endemic in South America, Southeast Asia and Africa and accounts for over 85% of the cases and much of the mortality from human malaria. A further problem is the widespread resistance of the anopheline vector to economical insecticides such as chlorophenothane (DDT). Furthermore, no major pharmaceutical company is known to have an active anti-malarial drug development program in place. Thus, as our need for new anti-malarial drugs becomes increasingly acute, the pipeline for such anti-malarial drugs appears to remain dry.

Current Anti-malarial Treatments.

The treatment of chloroquine-susceptible malaria (P. vivax, P. ovale or P. malariae malaria and chloroquine-susceptible P. falciparum malaria) is satisfactory because chloroquine is a safe and effective antimalarial. Chloroquine phosphate (ARALEN®) is available in tablet form for oral administration and, for severe cases of malaria, chloroquine hydrochloride is available as a solution for intravenous administration. Some patients with chloroquine-resistant P. vivax have been treated successfully with 1500 mg chloroquine base (25 mg base per kilogram) orally after failing to respond to 600 mg base, whereas others have required treatment with mefloquine. However, the treatment of chloroquine-resistant P. falciparum malaria is unsatisfactory using this same protocol.

Potential choices for treating chloroquine-resistant P. falciparum include quinidine gluconate, quinine sulfate, mefloquine hydrochloride (LARIAM®) and halofantrine (HALFAN®). Oral quinine therapy is combined with either tetracycline, pyrimethamine-sulfadoxine (FANSIDAR®), pyrimethamine (DARAPRIM®) and sulfadiazine, or doxycycline hyclate (VIBRAMYCIN®, and others). For a more complete summary of malaria treatments, see Krogstad, 1996, Malaria, page 1896, Table 374-3, In Cecil Textbook of Medicine, Bennett and Plum; Tracy and Webster, 1996, Drugs Used in the Chemotherapy of Protozoal Infections: Malaria, Chapter 40:965-985, In Goodman & Gilman's The Pharmacological Basis of Therapeutics, Ninth Edition, McGraw-Hill; and Berkow et al. (eds.), 1992, The Merck Manual, Sixteenth Edition, Chapter 15:229-232.

The Use of Triazines to Treat Apicomplexan Infections

Triazines are chemical compounds of general formula C₃H₃N₃ where the three carbon and three nitrogen atoms form a six-membered ring. There are three possible isomers of triazine. Triazines and triazine derivatives have achieved a relatively high degree of commercial success as herbicidal compounds (see, e.g., Hance et al., (eds.), 1990, Weed Control Handbook: Principles, Blackwell Science Inc. and Roe et al. (eds.), 1997, Herbicide Activity: Toxicology, Biochemistry and Molecular Biology, IOS Press).

Triazines and triazine derivatives have been used in anti-malarial and anti-bacterial compounds and compositions (see, e.g., U.S. Pat. Nos.: 1,217,415; 3,215,600; 3,272,814; 3,632,583; 3,632,762; 3,666,757; 3,637,688; 3,666,757; 3,723,429; 3,876,785; 4,035,146; 5,188,832).

Triazines and triazine derivatives control Apicomplexans by inhibiting dihydrofolate reductase (DHFR) (Matthews et al., 1985, J. Biol. Chem. 260(1):392-399; Dedhar et al., 1986, Biochem. Pharmacol. 35(7):1143-1147; Yeo et al., 1997, Biochem. Pharmacol. 53(7):943-950). Thus, triazine compounds are usually classified as antifolates. As mentioned previously, antifolate resistant strains of P. falciparum are becoming ubiquitous.

The Need For New Anti-Malarial Treatments.

As with antibiotics, the extensive use of antimalarials has expedited the selection of drug-resistant strains of P. falciparum. Since 1960, transmission of malaria has risen in most regions where the infection is endemic, chloroquine-resistant and multidrug-resistant strains of P. falciparum have spread, and the degree of drug resistance has increased. For a recent review of various mechanisms of plasmodial resistance to antimalarial drugs, see van Es et al., 1993, Chemotherapy of malaria: a battle against all odds?, Clin. Invest. Med. 16:285-293.

Except for parts of Africa, there is extensive geographic overlap between chloroquine resistance and resistance to pyrimethamine-sulfadoxine, a combination of antifolate drugs formally used extensively for chemoprophylaxis of falciparum malaria. Multidrug resistance now extends to effective but more toxic blood schizontocides such as quinine and, more recently, mefloquine and halfantrine. Increased doses of these agents often are required to treat falciparum malaria despite the enhanced risk of dose-related toxicity. The increasing prevalence of multidrug resistance dramatically illustrates the continuous need for new antimalarial agents.

Drug resistant forms of Apicomplexan parasites other than P. falciparum are also becoming more commonplace. For example, chloroquine-resistant forms of Plasmodium berghei are cross-resistant to related drugs, including amodiaquine, quinine and mefloquine (Platel et al., 1998, Int. J. Parasitol. 28(4):641-651). Chloroquine-resistant strains of P. vivax are also becoming more common and more difficult to treat.

Object of the Invention

Practical, effective and safe compounds are urgently needed to combat malaria and other infectious diseases caused by Apicomplexans. Present compounds, such as chloroquine and triazine, are rapidly becoming obsolete, while the development of new antimalarials, such as the endoperoxides, has not kept pace with the need. The present invention provides an alternative family of compounds useful for the safe, economic and effective treatment of infections caused by Apicomplexan parasites, including the causal agent of falciparum malaria. The compounds and methods of the present invention provide an alternative therapeutic option since they target non-antifolate activity in controlling Apicomplexans. Thus, the new compounds disclosed herein are useful for the treatment of antifolate-resistant strains of Apicomplexans.

### SUMMARY OF THE INVENTION

This invention comprises pharmaceutical compositions and methods of using such compositions for the treatment of infections caused by parasites. More specifically, this invention provides pharmaceutical compositions and methods utilizing such compositions for treating infections in humans and animals by any Apicomplexan parasite. Examples of parasitic infections which may be successfully treated using the pharmaceutical compositions of the present invention include those resulting from infection by Plasmodium sp, Toxoplasma sp, Neospora sp, Cryptosporidium sp, Hematodinium sp, Hemogregarines sp, Babesia sp, Eimeria sp, and Theileria sp. Even more specifically, this invention provides pharmaceutical compositions and methods utilizing such compositions effective for treating infections by the malarial parasite Plasmodium falciparum.

The pharmaceutical compositions used in the methods of the present invention include one or more s-triazine compounds and a pharmaceutically acceptable carrier. In a preferred embodiment of the present invention, the s-triazine compound is 2-chloro-4-ethylamino-6-isopropylamino-s-triazine, more commonly known as atrazine. In another preferred embodiment of the present invention, the s-triazine compound is 2-chloro-4,6-di(isopropylamino)-s-triazine, more commonly known as propazine. In yet another preferred embodiment of the present invention, the s-triazine compound is 2-chloro-4,6-di(ethylamino)-s-triazine, more commonly known as simazine. Pharmaceutically acceptable salts of the s-triazine compounds are contemplated for use in the present invention as well. Preferably, pharmaceutically acceptable salts of the s-triazine compounds are those salts of the C2, C4, and/or C6 groups of the s-triazine compounds, as described herein.

The present invention provides pharmaceutical compositions and methods utilizing such compositions effective for treating any human or animal infected by an Apicomplexan parasite. The present invention also provides pharmaceutical compositions and methods utilizing such compositions effective for the prophylactic treatment of any human or animal before infection by an Apicomplexan parasite. Examples of animals which may be successfully treated using the pharmaceutical compositions of the present invention include, but are not limited to, guinea pigs, dogs, sheep, cattle, horses, pigs, cats, goats, rats, mice, and hamsters as well as chickens, ducks, turkeys and other fowl.

The present invention also contemplates s-triazine compositions and methods of utilizing such compositions for the treatment of humans and animals infected with Apicomplexans which are resistant to currently-used drugs. More specifically, the s-triazine compositions of the present invention can be used to treat infections caused by antifolate-resistant Apicomplexans and chloroquine-resistant Apicomplexans. The anti-parasitic activities of the s-triazines of the present invention are different than the antifolate activity of triazines, wherein the triazines do not have the same chemical structure as the s-triazines of the present invention.

The present invention further contemplates pharmaceutical compositions which combine s-triazines with other compounds having therapeutic and/or prophylactic activity, particularly anti-biocidals. More specifically, the present invention contemplates combining s-triazines with compounds which have a different mode of anti-parasitic activity than the s-triazines. Even more specifically, the present invention contemplates combining s-triazines with other compounds, particularly anti-malarials, such as chloroquine, mefloquine and quinine, as well as other herbicide compounds known to have anti-malarial activity, e.g., glyphosate.

One skilled in the art can easily make any necessary adjustments in accordance with the necessities of the particular situation.

Further objects and advantages of the present invention will be clear from the description that follows.

### BRIEF DESCRIPTION OF THE DRAWINGS

FIG. 1. FIG. 1 depicts the percentage of Red Blood Cells (RBCs) infected with Plasmodium falciparum 48 hrs after treatment with different types and concentrations of chemical compounds. x1=the compound was present for the first 24 hour period only. x3=the compound was added at each change of the medium.

FIG. 2. FIG. 2 depicts the percentage of Red Blood Cells (RBCs) infected with Plasmodium falciparum 24 hrs after treatment with different types and concentrations of chemical compounds.

FIG. 3. FIG. 3 depicts the percentage of Red Blood Cells (RBCs) infected with Plasmodium falciparum 96 hrs after treatment with different types and concentrations of chemical compounds.

FIG. 4. FIG. 4 depicts the percentage of Red Blood Cells (RBCs) infected with Plasmodium falciparum 96 hrs after treatment with different types and concentrations of chemical compounds.

FIG. 5. FIG. 5 depicts the RBC infection percentage following treatment with either atrazine or chloroquine at concentrations ranging from 0.006 μM to 0.4 μM.

FIG. 6. FIG. 6 depicts the RBC infection percentage following in vivo treatment with either atrazine or chloroquine at concentrations of 20 mg/kg.

FIG. 7. FIG. 7 depicts yeast growth as a percentage of the control following no treatment (control) or following treatment with atrazine, chloroquine (Clq) or pyrimethamine (pyr). The yeast types tested included wild type, folate sensitive (folate sen) and folate insensitive (folate insen).

FIG. 8. FIG. 8 depicts the percentage of Red Blood Cells (RBCs) infected with Plasmodium falciparum 48 hrs after either no treatment (No Drug) or after treatment with atrazine or chloroquine (Clq). The P. falciparum types tested included wild type, mefloquine resistant (Mfl res), chloroquine resistant (Clq res) and multi-drug resistant (MDR).

FIG. 9. FIG. 9 depicts the percentage of Red Blood Cells (RBCs) infected with Plasmodium falciparum 96 hrs after either no treatment (No Drug) or after treatment with atrazine or chloroquine (Clq). The P. falciparum types tested included wild type, mefloquine resistant (Mfl res), chloroquine resistant (Clq res) and multi-drug resistant (MDR).

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Overview of the Invention

As discussed above, there is an immediate need for new, non-antifolate compounds to treat Apicomplexan infections, including malaria. As described below, the present inventors have unexpectedly discovered that s-triazine compounds satisfy this need.

S-Triazines.

This invention describes a new use for an established family of chemical compounds known as s-triazines, several of which are used presently as herbicides. This class of chemical compounds has been effective in controlling unwanted weed growth for more than 25 years. See for example, U.S. Pat. Nos. 3,787,199 and 3,925,055, both of which are herein incorporated by reference in their entirety. The compounds work as herbicides by interfering with energy production by the plant through inhibition of chloroplast function in the plant. The chemical compounds bind to and inhibit a protein found only in chloroplasts and as a result they are extraordinarily selective for organisms containing chloroplasts. The 2-chloro-4,6-diamino-s-triazines have also been utilized as algicides (see U.S. Pat. No. 4,659,359).

Two recent papers (that are not prior art to the present invention) have indicated that the Apicomplexan plastids may be good drug targets and that Apicomplexan growth may be inhibited by using appropriate herbicides; see, Zuther et al., Nov. 9, 1999, PNAS 96(23:13387-13392 and McFadden et al., August, 1999, Trends in Microbiology 7(8):328-333, both of which were published after the effective priority document filing dates of the present application and both of which are hereby incorporated by reference in their entireties.

Organisms lacking chloroplasts, which includes most bacteria, fungi and animals are relatively unaffected by these chemical compounds. In fact, the LD₅₀ (the dose of chemical compound required to kill 50% of the animals treated) for atrazine in rats given a single injection of the chemical compound is in the range of 3000 mg/kg (on par with table salt).

More particularly, this invention relates to the use of at least one C-substituted s-triazine derivative as inhibitors of Apicomplexans. As used herein, the term “s-triazine” refers to a 1,3,5-triazine, a symmetrical aromatic six-membered ring of general formula (I):

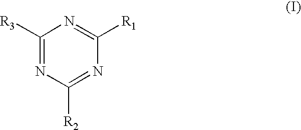

or a pharmaceutically acceptable salt thereof. As described below, s-triazines for use in the present invention may be substituted at at least one of the C2, C4 and C6 positions. The loss of one of the three double bonds in the s-triazine ring produces a compound that would not be considered a “s-triazine” for the purposes of this invention. For example, U.S. Pat. No. 3,272,814 describes 4,6-diamino-1-aryl-1,2-dihydro-s-triazines and methods of using such compounds as biocides. Since the triazine ring of the compounds disclosed in U.S. Pat. No. 3,272,814 contains only two double bonds, such a compound would not be considered a “s-triazine” as defined herein. In addition, 1,3,5-triazines N-substituted at the 1, 3, and/or 5 position would not be considered a “s-triazine” for the purposes of this invention, since such N-substitution would result in loss of aromaticity of the compound. Examples of such N-substituted 1,3,5-triazine compounds can be found in U.S. Pat. Nos. 3,933,814; 3,948,893; 3,966,725; 3,970,752; 4,219,552; 4,631,278; 4,826,842; 4,837,216; 4,912,106; 4,935,423; 4,952,570; 4,968,795; 5,114,938; 5,141,938; 5,188,832; 5,196,562; 5,214,043; 5,219,853; 5,256,631; 5,464,837; 5,624,678; 5,646,135; 5,830,893; 5,834,473; 5,876,780; and 5,883,095, each of which is incorporated in its entirety by reference. Diclazuril, a benzeneacetonitrile, and toltrazuril, a symmetrical triazinone derivative, have been found to be effective against a broad spectrum of protozoan parasites (Haberkorn, 1996, Parasitol. Res. 82:193-199; Armson et al., 1999, FEMS Microbiology Letters 178:227-233; Mehlhorn et al., 1988, Parasitol. Res. 75:64-66; Hackstein et al., 1995, Parasitol. Res. 81:207-216). Since diclazuril and toltrazuril each have N-substitutions at the 5 positions (The Merck Index, Twelfth Edition, 1996, Merck Research Laboratories, pp. 3133 and 9665, respectively), these compounds would not be considered “s-triazines” for the purposes of this invention.

Furthermore, as described elsewhere herein, the s-triazines of the present invention are believed to have a different mode of anti-parasitic action than previously tested triazine compounds. While not wishing to be bound by any particular theory or mechanism of action, the inventors believe that triazine compounds having aromatic substituents provide DHFR inhibition. Thus, the dominant mode of action of the s-triazines of the present invention is not antifolate inhibition. As stated previously, the anti-parasitic activities of the s-triazines of the present invention are different than the antifolate activity of triazines, wherein the triazines do not have the same chemical structure as the s-triazines of the present invention.

The s-triazines used in the present invention may be any s-triazine (as defined above) known in the art including those described in U.S. Pat. Nos. 2,385,766; 2,867,621; 2,394,526; 2,463,471; 3,086,855; 3,162,633; 3,305,390; 3,530,121; 3,536,708; 3,553,326; 3,816,419; 3,932,167; 4,680,054; 4,844,731; 4,874,420; 4,932,998; 5,250,685; 5,250,686; 5,290,754; and 5,403,815; and WO 96/25404;,WO 97/00254; WO 97/08156; WO 98/15536; WO 98/15537; WO 98/15538; WO 98/15539; WO 99/19309, each of which is incorporated in their entirety by reference. Pharmaceutically acceptable salts of the s-triazines are contemplated by the invention as well. Preferably, a s-triazine for use in the pharmaceutical compositions and methods of use of such compositions of the invention is of formula (I):

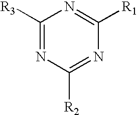

or a pharmaceutically acceptable salt thereof. In formula (I), R₁, R₂, and R₃ are, independently:

hydrogen;

halogen;

an optionally substituted, linear or branched C₁-C₂₀ alkyl, preferably, C₁-C₁₂ alkyl, more preferably, C₁-C₆ alkyl group;

an optionally substituted, linear or branched C₂-C₂₀ alkenyl, preferably, C₂-C₁₂ alkenyl, more preferably, C₂-C₆ alkenyl group;

an optionally substituted, linear or branched C₂-C₂₀ alkynyl, preferably, C₂-C₁₂ alkynyl, more preferably, C₂-C₆ alkynyl group;

an optionally substituted, C₃-C₁₂ cycloalkyl, preferably, C₃-C₈ cycloalkyl, more preferably C₃-C₆ cycloalkyl group;

an optionally substituted, C₆-C₂₀ aryl, preferably, C₆-C₁₅ aryl, more preferably C₆-C₁₂ aryl group;

an optionally substituted, C₃-C₁₂ heterocyclic, preferably, C₃-C₈ heterocyclic, more preferably, C₃-C₆ heterocyclic group containing at least one heteroatom of nitrogen (N), oxygen (O), or sulfur (S);

OR₄; SR₄; NO₂; NR₄R₅; N═CHR₄; NR₄C(O)R₄; C(O)R₄; C(O)OR₄; or CN. R₄ and R₅ are, independently:

hydrogen;

an optionally substituted, linear or branched C₁-C₂₀ alkyl, preferably, C₁-C₁₂ alkyl, more preferably, C₁-C₆ alkyl group;

an optionally substituted, linear or branched C₂-C₂₀ alkenyl, preferably, C₂-C₁₂ alkenyl, more preferably, C₂-C₆ alkenyl group;

an optionally substituted, linear or branched C₂-C₂₀ alkynyl, preferably, C₂-C₁₂ alkynyl, more preferably, C₂-C₆ alkynyl group;

an optionally substituted, C₃-C₁₂ cycloalkyl, preferably, C₃-C₈ cycloalkyl, more preferably C₃-C₆ cycloalkyl group;

an optionally substituted, C₆-C₂₀ aryl, preferably, C₆-C₁₅ aryl, more preferably C₆-C₁₂ aryl group;

an optionally substituted, C₃-C₁₂ heterocyclic, preferably, C₃-C₈ heterocyclic, more preferably, C₃-C₆ heterocyclic group containing at least one heteroatom of N, O, or S;

CN group;

or R₄ and R₅ when taken together with N forms a heterocyclic group According to the invention, R₁, R₂, and R₃ cannot all be hydrogen or all CN groups.

The term “halogen” as used herein refers to any halo group (e.g. fluoro, chloro, bromo, and iodo) as recognized by those of skill in the art. Preferably, at least one of R₁, R₂, and R₃ of formula (I) is a halogen. More preferably, at least one of R₁, R₂, and R₃ is a chloro group.

The term “alkyl group” as used herein refers to a saturated hydrocarbon chain. Examples of suitable alkyl groups include, but are not limited to, methyl, ethyl, propyl, isopropyl, n-butyl, isobutyl, sec-butyl, tert-butyl, amyl, isoamyl, and hexyl.

The term “alkenyl group” as used herein refers to a hydrocarbon chain containing at least one double bond. Examples of suitable alkenyl groups include, but are not limited to, ethenyl, propenyl, butenyl, pentenyl, and hexenyl.

The term “alkynyl group” as used herein refers to a hydrocarbon chain containing at least one triple bond. Examples of suitable alkynyl groups include, but are not limited to, ethynyl, propynyl, butynyl, pentynyl, and hexynyl.

The term “cycloalkyl group” as used herein refers to a cyclic aliphatic hydrocarbon. Examples of suitable cycloalkyl groups include, but are not limited to, cyclopropyl, cyclobutyl, cyclopentyl, and cyclohexyl.

The term “aryl” group as used herein refers to an aromatic mono-, bi- or polycyclic ring system which optionally contains at least one heteroatom of nitrogen (N), oxygen (O), or sulfur (S). Examples of suitable aryl groups include, but are not limited to, phenyl, napthyl, anthryl, phenanthryl, and pyridinyl.

The term “heterocyclic group” as used herein refers to a saturated or unsaturated, non-aromatic mono-, bi- or polycyclic ring system containing at least one heteroatom of nitrogen (N), oxygen (O), or sulfur (S). Examples of suitable heterocyclic groups include, but are not limited to, aziridino, piperidino, morpholino, piperazino, N′-alkylpiperazino, N′-alkanolpiperazino.

The term “pharmaceutically acceptable salt(s)” as used herein refers to those salt(s) of the s-triazine compounds of the present invention which retain the pharmaceutical or biological effectiveness and properties of the neutral s-triazine compound and which are not pharmaceutically, biologically or otherwise undesirable.

According to the invention, R₁, R₂, R₃, R₄, and R₅, each as described above, may be further substituted with at least one substituent. Suitable “substituents” include those recognized by those of skill in the art. Examples of suitable substituents include, but are not limited to, halogen, hydroxy (—OH), alkoxy (e.g. —OR₄), oxyaryl (e.g. R₄OAr—), aryloxy (e.g. —OAr), carboxylic (e.g. —CO₂H), sulfonic (e.g. —SO₃H), carboxylate (e.g. —C(O)OR₄), carbonyl (e.g. —C(O)R, —C(O)H), amino (e.g. —NR₄R₅), amido (e.g. —C(O)NR₄R₅), thioalkyl (e.g. SR₄), the same or different s-triazine of formula (I), as well as those substituents described in U.S. Pat. Nos. 2,385,766, 2,867,621, 2,394,526, 2,463,471, 3,086,855, 3,162,633, 3,305,390, 3,530,121, 3,536,708, 3,553,326, 3,816,419, 3,932,167, 4,680,054, 4,844,731, 4,874,420, 4,932,998, 5,250,685, 5,250,686, 5,290,754, and 5,403,815; and WO 96/25404, WO 97/00254, WO 97/08156, WO 98/15536, WO 98/15537, WO 98/15538, WO 98/15539, WO 99/19309, each of which is incorporated in their entirety by reference.

In a preferred aspect of the invention, a s-triazine for use in the invention is a s-triazine of formula (I) where R₂ and R₃ are both a —NH₂ group, as shown in formula (II), or where R₂ is —NHR₄ and R₃ is a —NH₂ group, as shown in formula (III):

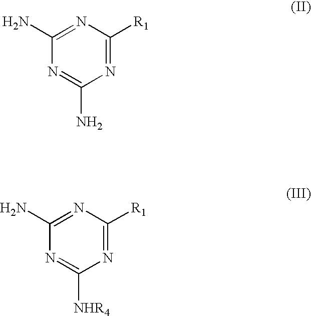

or a pharmaceutically acceptable salt thereof. In formulae (II) and (III), R₁ and R₄ are each as described above.

In another preferred aspect of the invention, in formula (I), R₁ is a chloro group and R₂ and R₃ are each the same or different NHR₄ group, where R₄ is as described above, as shown in formula (IV):

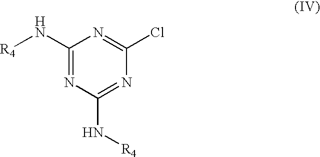

or a pharmaceutically acceptable salt thereof. Preferably, in formula (IV), each R₄ may be the same or different substituted or unsubstituted, linear or branched C₁-C₅ alkyl group.

In a more preferred aspect of the invention, in formula (I), R₁ is a chloro group, R₂ is NHCH₂CH₃, and R₃ is NHCH(CH₃)₂, i.e. 2-chloro-4-ethylamino-6-isopropylamino-s-triazine, more commonly known as atrazine, of formula (V):

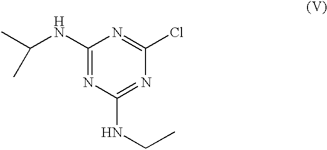

or a pharmaceutically acceptable salt thereof.

For a complete listing of the various other names used to refer to atrazine as well as thermochemistry data, phase change data, gas phase IR spectrum and mass spectrum, see The National Institute of Standards and Technology (NIST) Standard Reference Database 69, March 1998 Release: NIST Chemistry; The Merck Index. Eleventh Edition, 886, p. 137 (Merck & Co., Inc., 1989); and the NIST chemistry website (http://webbook.nist.gov/cgi/book.exe?Name+atrazine&Unites=SI).

In another more preferred aspect of the invention, in formula (I), R₁ is a chloro group and R₂ and R₃ are both a NHCH(CH₃)₂ group, i.e. 2-chloro-4,6-di(isopropylamino)-s-triazine, more commonly known as propazine, of formula (VI) or are both a NHCH₂CH₃ group, i.e. 2-chloro-4,6-di(ethylamino)-s-triazine, more commonly known as simazine, of formula (VII):

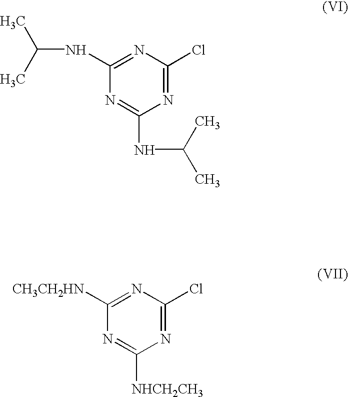

or a pharmaceutically acceptable salt thereof.

In still another more preferred embodiment of the invention, in formula (I), R₁ is a chloro group, R₂ is a NHCH₂CH₃ group and R₃ is a NHC(CN)(CH₃)₂ group, i.e. 2-[[4-chloro-6-(ethylamino)-s-triazin-2-yl]amino]-2-methyl propionitrile, more commonly known as cyanazine, of formula (VIII):

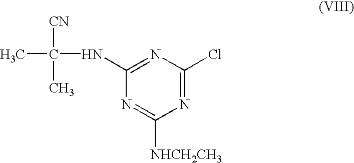

or a pharmaceutically acceptable salt thereof.

Still further, in a more preferred embodiment of the invention, in formula (I), R₁ is a —SCH₃ group, R₂ is NHCH(CH₃)₂, and R₃ is NHCH₂CH₃, i.e. 2-ethylamino-4-isopropylamino-6-thiomethoxy-s-triazine, more commonly known as ametryn, of formula (IX):

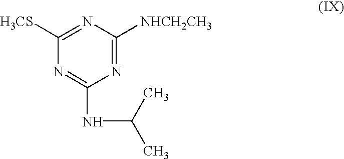

or a pharmaceutically acceptable salt thereof.

Pharmaceutical Preparations.

According to the invention, a pharmaceutical composition for use in the treatment or prevention of mammalian infection by a parasite of the phylum Apicomplexa comprises a therapeutically effective amount of at least one s-triazine compound or a pharmaceutically acceptable salt thereof. According to the invention, a pharmaceutical composition may further include a pharmaceutically acceptable carrier.

Examples of parasitic infections which may be successfully treated using the pharmaceutical compositions of the present invention include, but are not limited to, those resulting from infection by Plasmodium sp, Toxoplasma sp, Neospora sp, Cryptosporidium sp, Hematodinium sp, Hemogregarines sp, Babesia sp, Eimeria sp, and Theileria sp. Even more specifically, parasitic infections caused by the malarial parasite Plasmodium falciparum may be treated or prevented with a pharmaceutical composition of the invention.

Also as recognized by one of skill in the art, a “therapeutically effective amount” will be determined on a case by case basis. Factors to be considered include, but are not limited to, the degree of infection, the physical characteristics of the one suffering from the infection, the route of administration of the pharmaceutical composition, and the parasite causing the infection. Accordingly, a “therapeutically effective amount” will be best determined through routine experimentation. In general, however, a “therapeutically effective amount” is any amount sufficient to treat or prevent infection by a parasite of the phylum Apicomplexa. Preferably, a therapeutically effective amount of the s-triazine compounds of the present invention is in the range of from about 0.01 to about 1000 mg of the active ingredient per kg of weight of the subject being treated (mg/kg), preferably from about 0.1 to about 1000 mg/kg. As discussed above, the actual dosages of the compounds are adjusted based on the degree of infection, the specific human or animal undergoing treatment, the route of administration and the specific parasitic organism(s) targeted for treatment. In addition, the actual dosage may be adjusted for any additional therapeutic compounds which may be administered before, during or after treatment with the compounds of the present invention.

The s-triazine compound is as described above. The pharmaceutically acceptable carrier may be any such carrier known in the art, preferably a pharmaceutically acceptable, non-toxic sterile carrier as would be recognized by one of skill in the art. See, for example, Remington's Pharmaceutical Sciences, 19th edition, Mack Publishing Company, 1995.

For oral administration, the s-triazine derivatives of the present invention may take the form of, for example, tablets or capsules prepared by conventional means in admixture with generally acceptable excipients such as binding agents (e.g., pregelatinised maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate); glidants; artificial and natural flavors and sweeteners; artificial or natural colors and dyes; and solubilizers. The s-triazine compositions may be additionally formulated to release the active agents in a time-release manner as is known in the art and as discussed in U.S. Pat. Nos. 4,690,825 and 5,055,300. The tablets may be coated by methods well known in the art.

Liquid preparations for oral administration of s-triazine compounds of the invention may take the form of, for example, solutions, syrups, suspensions, or slurries (such as the liquid nutritional supplements described in U.S. Pat. No. 5,108,767), or they may be presented as a dry product for reconstitution with water or other suitable vehicles before use. Liquid preparations of s-triazine compounds of the invention, and other vitamins and minerals may come in the form of a liquid nutritional supplement designed for the specific therapeutic needs of patients. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

For buccal administration, the composition may take the form of tablets or lozenges formulated in conventional manners.

The s-triazine compounds of the invention may be formulated for parenteral administration by injection, which includes using conventional catheterization techniques or infusion. Compositions for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredients may be in powder form for reconstitution with a suitable vehicle, e.g., sterile pyrogen-free water, before use.

The active compounds may also be formulated in rectal compositions such as suppositories or retention enemas, e.g., containing conventional suppository bases such as cocoa butter or other glycerides.

For intranasal administration or administration by inhalation, the s-triazine is conveniently delivered in the form of a solution or suspension from a pump spray container that is squeezed or pumped by the patient, or as an aerosol spray presentation from a pressurized container or nebulizer, with the use of a suitable propellant (e.g., dichlorodifluoromethane, trichlorofluoromethane, dichlorotetrafluoroethane, carbon dioxide or other suitable gas). In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of an active compound and a suitable powder base such as lactose or starch.

For intravenous administration (IV), s-triazine, its analogs, derivatives, as well as other vitamins, minerals, homocysteine-modulating agents and antioxidants will be administered as an IV admixture in a suitable isotonic vehicle.

The s-triazine compounds utilized in any of the above preparations may be combined with one or more additional or supplemental compounds, particularly anti-parasitic drugs. General examples of supplemental compounds useful in the present invention include triazines, chloroquine, quinidine, quinine, mefloquine, doxycycline, chloroguanide, tetracycline, pyrimethamine and halofantrine. Specific examples of supplemental compounds useful in the present invention include chloroquine phosphate (ARALEN®), primaquine phosphate, mefloquine hydrochloride (LARIAM®), pyrimethamine-sulfadoxine (FANSIDAR®), doxycycline hyclate (VIBRAMYCIN®), chloroguanide hydrochloride (proguanil; PALUDRINE®), quinine sulfate, pyrimethamine (DARAPRIM®) and sulfadiazine, and halofantrine (HALFAN®).

The composition and administration of triazine compounds for the control of parasitic infections in humans and animals are well known to one skilled in the art of pharmaceutical preparation and administration (see, e.g., U.S. Pat. Nos.: 1,217,415; 3,215,600; 3,272,814; 3,632,583; 3,632,762; 3,663,693, 3,666,757; 3,637,688; 3,723,429; 3,876,785; 4,035,146; 5,188,832). The known compositions and administration of these triazine compounds can be used as guidelines in the preparation of compositions containing atrazine or other s-triazines of the present invention as well as the administration of compositions containing atrazine or other s-triazine compounds.

In addition to s-triazines, other chloroplast poisons may be used to treat infections caused by parasites such as an Apicomplexan parasite. Such compounds include, but are not limited to, phenylureas and uracils including their analogs and derivatives. Preferably, phenylureas, uracils, their analogs and derivatives, target non-antifolate activity in controlling parasites such as Apicomplexans. A person of skill in the art would be able to evaluate or determine the effectiveness of a particular chloroplast poison in the treatment of infections caused by parasites such as an Apicomplexan parasite by using the assay as described, for example, in Example 1.

Without further description, it is believed that one of ordinary skill in the art, using the preceding description and the following illustrative examples, can make and utilize the compounds of the present invention and practice the claimed methods.

### EXAMPLES

### Example 1

### In Vitro Evaluation of Atrazine Against Plasmodium falciparum

The in vitro method used to test the anti-malarial effectiveness of various s-triazines was adapted from Trager, 1994, Methods in Cell Biology Vol 45:7-26.

Briefly, a red blood cell suspension at a malarial (wild type Plasmodium falciparum) infection rate of approximately 1% was added to each well of a 96 well micro titer plate. The test compound was added to the cultures at final concentrations of 2.0, 0.2 or 0.02 μM. and incubated in an environment of 5% CO₂, 5% O₂ and 90% N₂ at 37° C. Every 24 hrs the dish was removed, 100 μl of spent medium removed and 100 μl of fresh, compound containing-medium was added back to each well. This procedure was repeated daily.

At 48 hrs and again at 96 hrs, blood smears were made from each well and 500 Red Blood Cells (RBCs) were counted and inspected for malarial infection. The percent infection rate was then determined for each experimental condition tested.

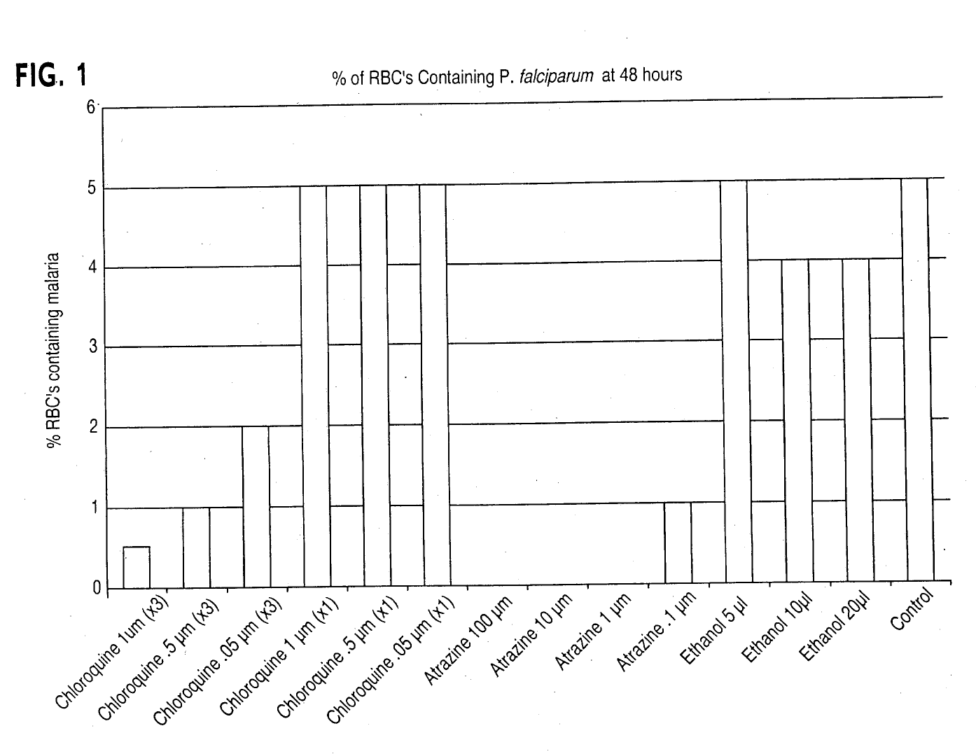

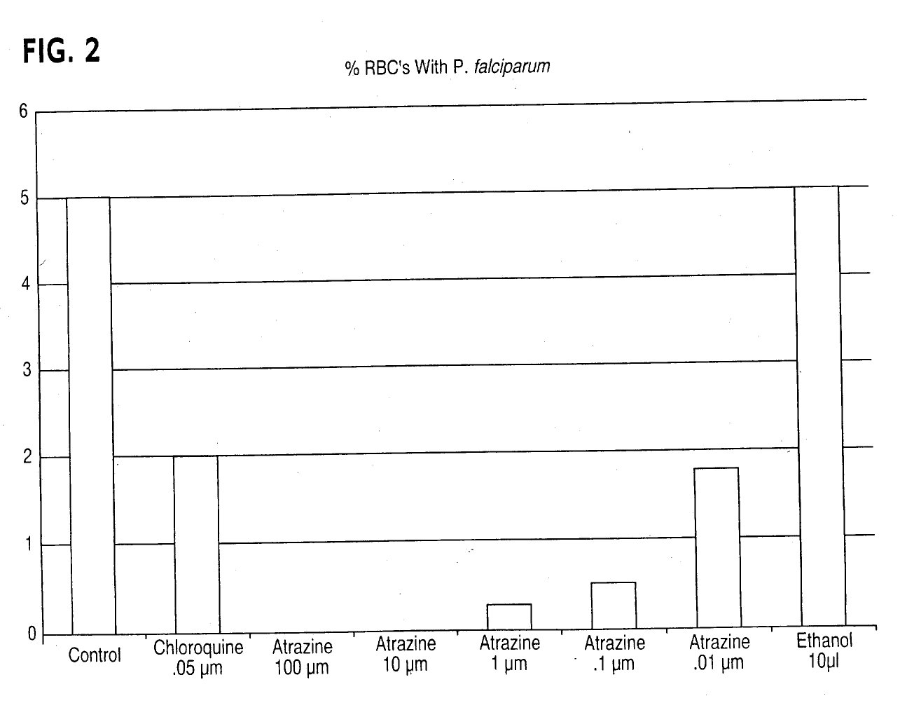

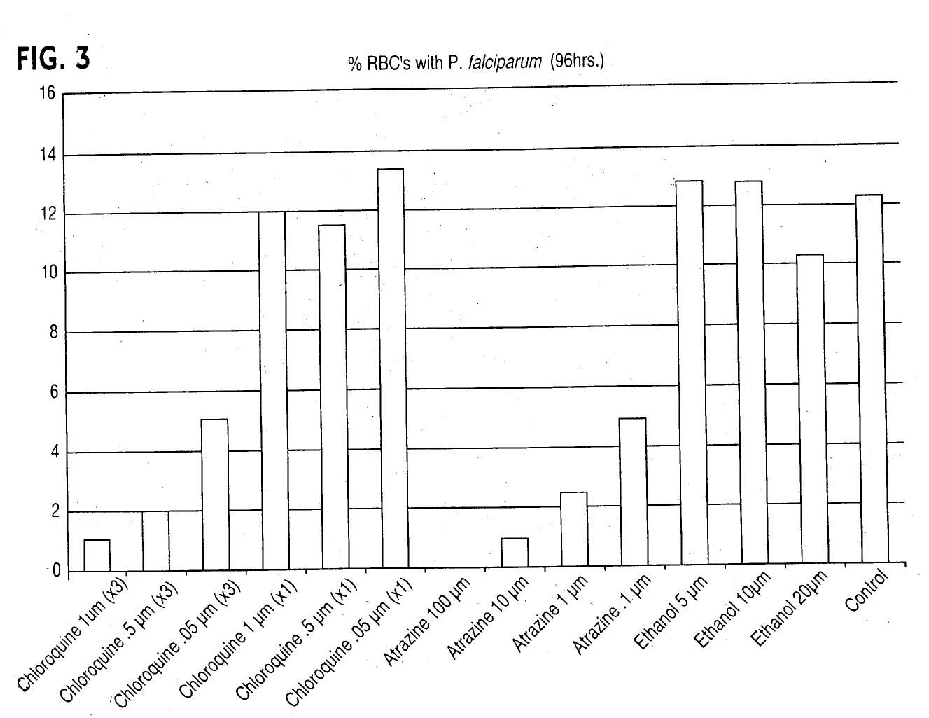

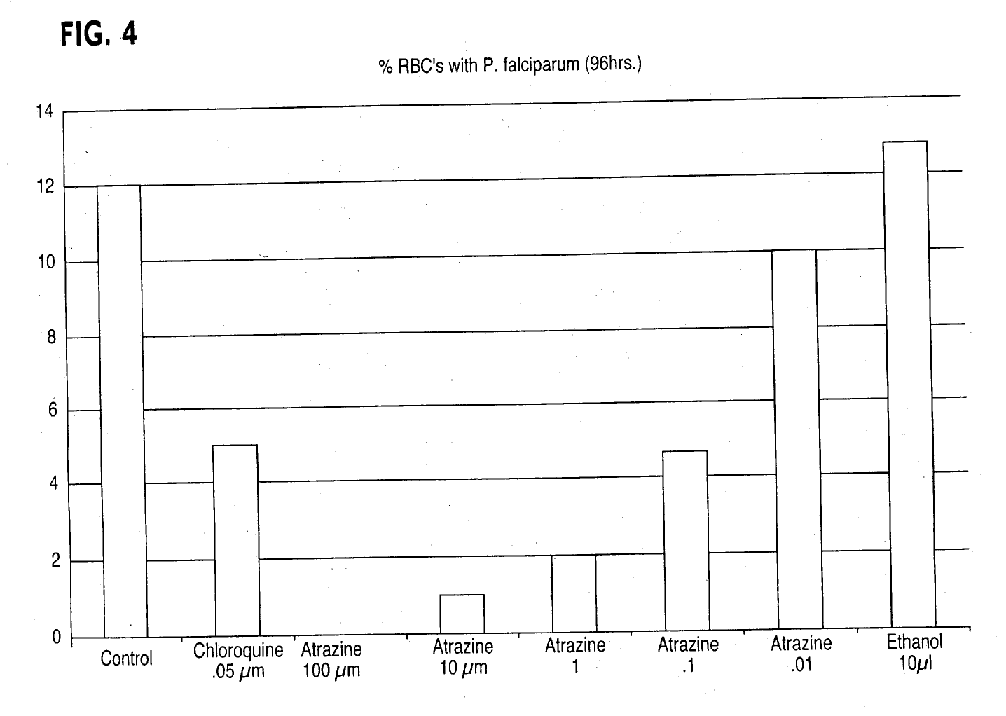

This test was repeated several times and the resulting data is presented in FIGS. 1-4. The figures show the percentage of RBCs infected with P. falciparum following treatment with various test compounds and for varying amounts of the test compounds. These data demonstrate that applying low concentrations of atrazine will result in a relatively low percentage of the RBCs being infected with P. falciparum at either 48 hrs or 96 hrs after treatment.

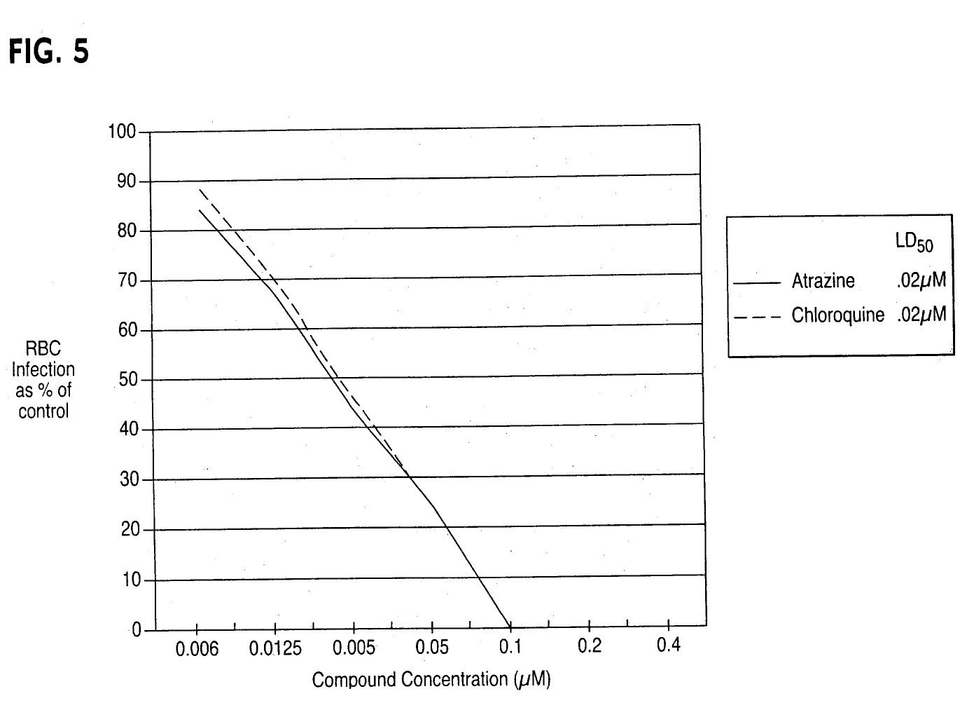

FIG. 5 displays a graphical comparison between the effectiveness of chloroquine and atrazine in reducing the percentage of RBCs infected with nonchloroquine-resistant, wild type P. falciparum. The figure shows that the RBC infection rates as a percent of the control are reduced at very similar rates as the concentration of atrazine and chloroquine are increased. These results demonstrate that atrazine is as effective and potent as chloroquine against P. falciparum wild type parasite.

### Example 2

### In Vitro Evaluation of Atrazine Against Chloroquine-Resistant P. falciparum

The procedure set forth in Example 1 is utilized for testing the effectiveness of chloroquine and airazine against chloroquine-resistant P. falciparum.

This experiment demonstrates that applying relatively low concentrations of atrazine to the red blood cell suspension will result in a relatively low percentage of the RBCs being infected with chloroquine-resistant P. falciparum at either 48 hrs or 96 hrs after treatment. In contrast, applying chloroquine to the chloroquine-resistant P. falciparum will result in a significantly higher level of RBCs being infected with the parasite.

### Example 3

### In Vitro Evaluation of Atrazine Against Mefloquine-Resistant P. falciparum

The procedure set forth in Example 1 is utilized for testing the effectiveness of mefloquine and atrazine against mefloquine-resistant P. falciparum.

This experiment demonstrates that applying relatively low concentrations of atrazine to the red blood cell suspension will result in a relatively low percentage of the RBCs being infected with mefloquine-resistant P. falciparum at either 48 hrs or 96 hrs after treatment. In contrast, applying mefloquine to the mefloquine-resistant P. falciparum will result in a significantly higher level of RBCs being infected with the parasite.

### Example 4

### In Vitro Evaluation of Atrazine Against Antifolate-Resistant P. falciparum

The procedure set forth in Example 1 is utilized for testing the effectiveness of triazine and atrazine against antifolate-resistant P. falciparum.

This experiment demonstrates that applying relatively low concentrations of atrazine to the red blood cell suspension will result in a relatively low percentage of the RBCs being infected with antifolate-resistant P. falciparum at either 48 hrs or 96 hrs after treatment. In contrast, applying triazine to the antifolate-resistant P. falciparum will result in a significantly higher level of RBCs being infected with the parasite.

### Experiment 5

### In Vitro Evaluation of Atrazine Against Plasmodium berghi

The procedure set forth in Example 1 is utilized for testing compounds against wild type rodent malaria (Plasmodium berghi).

This experiment demonstrates that applying low rates of atrazine will result in a relatively low percentage of the RBCs being infected with P. berghi at either 48 hrs or 96 hrs after treatment.

### Experiment 6

### In Vivo Evaluation of Atrazine Against Plasmodium berghi

In vivo testing was performed using juvenile female Lewis rats. Rats were inoculated intraperitoneally with 30,000,000 Plasmodium berghi infected RBCs. Rats were bled daily from the tail vein and, when parasitemia reached 1-3%, therapy was begun.

Rats were treated through intravenous injection (I.V.) with either 0.2 ml of saline solution, 20 mg/kg chloroquine solution or 20 mg/kg atrazine solution.

Blood smears were obtained at 24 and 48 hrs. The percent RBCs infected was then calculated after counting and inspecting 500 RBCs for each rat.

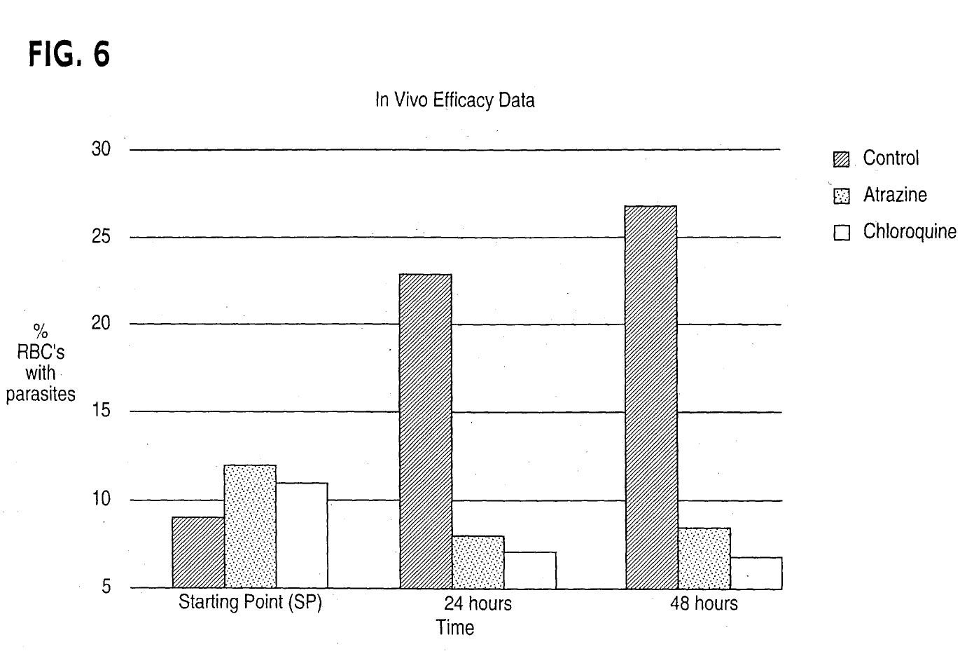

FIG. 6 displays a graphical comparison between the effectiveness of atrazine and chloroquine in reducing the in vivo percentage of RBCs infected with P. berghi. As shown in the figure, animals treated with either chloroquine or atrazine had their disease dramatically reduced compared to non-treated controls 24 and 48 hrs later.

### Example 7

### In Vitro Testing For Anti-Toxoplasmodium Activity of Atrazine

Human foreskin fibroblasts are grown to confluency in 12 well plates. Next, Toxoplasma gondii is added to the plates. The resultant cultures are treated with the test compound 24 hrs later. The percent infected fibroblasts is calculated 48 hrs after initiation of treatment.

This experiment demonstrates that applying atrazine to the fibroblast cells results in a relatively low percentage of the cells being infected with T. gondii 24 hrs after treatment.

### Example 8

### Testing for Antifolate Activity

We have established a in vitro yeast assay model for determining sensitivity to antifolate anti-malarials.

Saccharomyces cerevisiae was grown in culture and after 48 hours of treatment as described below the growth of the yeast was determined. Control (i.e., yeast grown with no treatment) is considered to be 100% growth. Drug treatment was then related to the controls as a percent of control growth. S. cerevisiae types tested included wild type yeast, yeast made sensitive to antifolate antimalarials by transfecting yeast with malarial dihydrofolate reductase (folate sensitive or folate sen), yeast transfected but not sensitive to antifolate antimalarials (folate insensitive or folate insen).

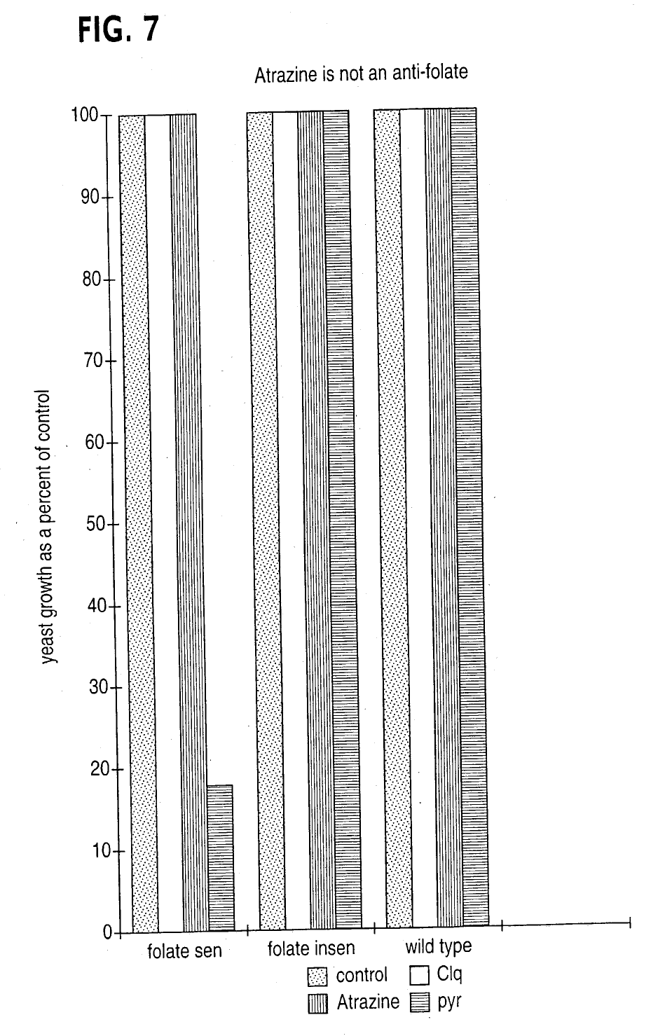

The results are presented in FIG. 7. These data demonstrate that pyrimethamine (pyr) (an antifolate antimalarial) does inhibit yeast growth of the folate sensitive yeast but not of the wild type yeast or the folate insensitive yeast. Neither chloroquine (Clq) nor atrazine affected growth of any of the yeast types tested. While this yeast assay system successfully identified pyrimethamine as active (i.e., as an antifolate), the assay indicated that both chloroquine (a non-antifolate) and atrazine lack antifolate activity. These data are important for they demonstrate that atrazine is NOT an antifolate, further supporting a novel mechanism of action for atrazine.

The ability of atrazine to inhibit dihydrofolate reductase (DHFR) is also tested using homogenates of calf liver, rat liver and cultures of P. falciparum. Atrazine's ability to inhibit mammalian DHFR (i.e., calf DHFR and rat DHFR) as well as malarial DHFR is determined using standard enzyme assays and the Michaelis-Menton analysis. The Michaelis-Menton hypothesis states that a complex is formed between an enzyme and its substrate and that the complex then dissociates to yield free enzyme and the reaction products, with the rate of dissociation determining the overall rate of substrate-product conversion. For a more complete review of the Michaelis constant, see Benet et al., 1996, Pharmacokinetics, Chapter 1:3-27, In Goodman & Gilman's The Pharmacological Basis of Therapeutics, Ninth Edition, McGraw-Hill; and Ross, 1996, Pharmacodynamics, Chapter 2:29-41, Id. The results of these DHFR assays indicate that atrazine does not inhibit DHFR.

The combined findings of these studies give a strong indication that atrazine has no antifolate activity.

### Example 9

### Evaluation of Interactions Between Atrazine and Other Anti-Malarials.

The procedure set forth in Example 1 is utilized for testing various combinations of different compounds against P. falciparum. Isobologram analysis will be utilized to compare the efficacy of the various compound combinations versus the efficacy of each compound being given by itself. Analysis of the data enables one to determine whether there is synergy, additivity, subadditivity or antagonism between the compounds.

### Example 10

### Additional Evaluations of Atrazine Against Drug-Resistant P. falciparum.

Malaria parasite (P. falciparum) was grown in culture as described previously. At 48 hours and 96 hours of treatment, red blood cells were taken from culture and the percent red blood cells infected with the parasite was determined by microscopic examination. The malarial parasites tested included wild type P. falciparum, chloroquine resistant P. falciparum (Clq res), mefloquine resistant P. falciparum (Mfl res) and multi-drug resistant P. falciparum (MDR).

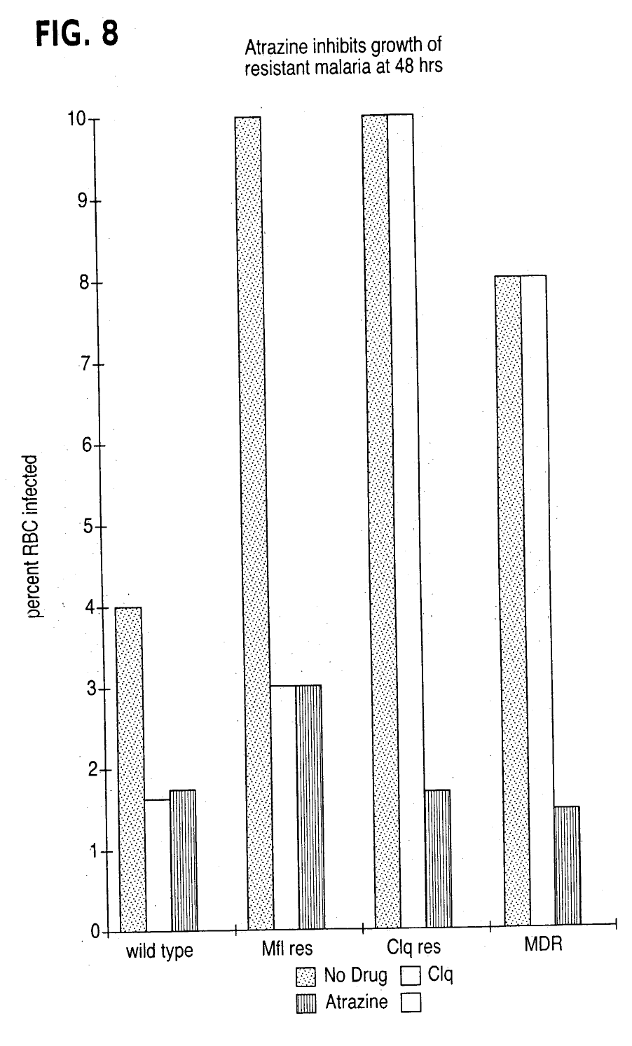

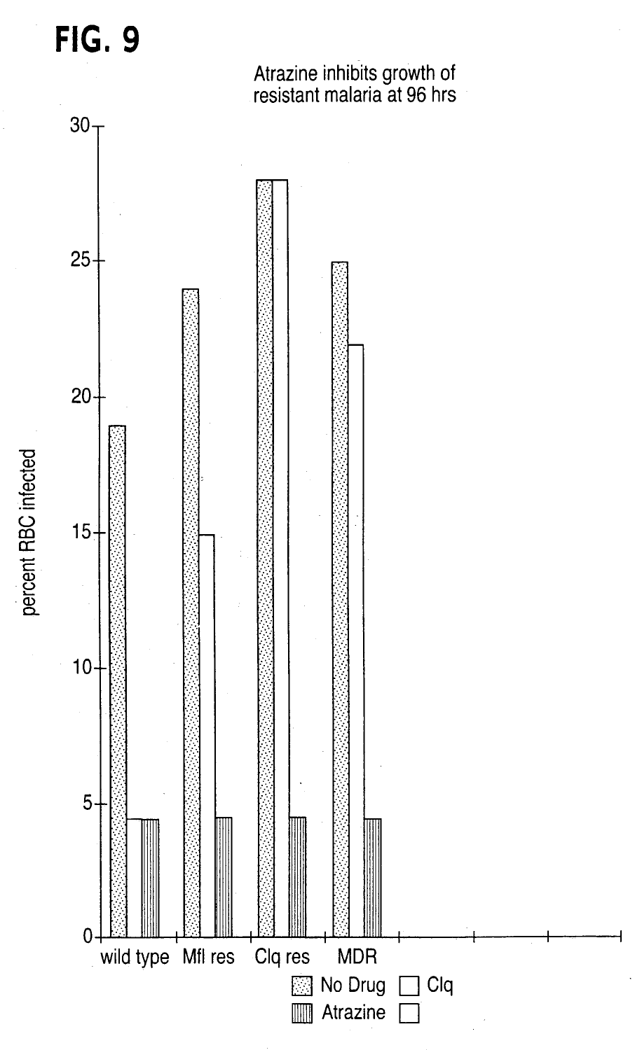

FIG. 8 shows data for the 48 hour evaluations and FIG. 9 shows the data for 96 hour evaluations. These data demonstrate that atrazine is effective against several classic forms of resistance developing in malaria. The fact that atrazine inhibits these parasites also indicates that atrazine does not kill the parasite through a mechanism related to chloroquine or mefloquine.

Following the same procedure, a subsequent evaluation of atrazine against drug-resistant P. falciparum was conducted; chloroquine was also tested for comparison. The red blood cell infection rate (%) at 96 hours of treatment is summarized in Table 1 below. These results, consistent with the results illustrated in FIG. 9, indicate the effectiveness of atrazine against several classic forms of malarial resistance.

TABLE 1
Red Blood Cell Infection Rate (%) at 96 Hrs of Treatment with Atrazine
(0.02 μM) and Chloroquine (0.02 μM).

| Malaria Phenotype | Control | Chloroquine | Atrazine |
|---|---|---|---|
| Wild Type | 18 | 3 | 3 |
| Mefloquine-resistant | 24 | 13 | 3 |
| Chloroquine-resistant | 28 | 28 | 4 |
| Multidrug-resistant | 25 | 22 | 3 |

### Example 11

### In Vitro Evaluation of s-Triazine Against Plasmodium falciparum

Following the procedure set forth in Example 1, the following antimalarial test compounds of cyanazine, propazine, ametryn, and simazine were evaluated and compared against atrazine, chloroquine and a control. The results are summarized in Table 2. The data in Table 2 represent percent red blood cell (RBC) infection rate at 48 hrs of treatment with the antimalarial test compound. Control cultures were 35% RBC infected. Propazine, simazine and atrazine were substantially similar in effectiveness as chloroquine. Ametryn and cyanazine were less effective.

TABLE 2
Red Blood Cell Infection Rate (%) at 48 Hrs of Treatment with an
Antimalarial Test Compound

| Drug | 2.0 μM | 0.2 μM | 0.02 μM |
|---|---|---|---|
| cyanazine | 16 | 17 | 18 |
| propazine | 1 | 1 | 5 |
| ametryn | 10 | 11 | 20 |
| simazine | 0 | 0 | 3 |
| atrazine | 0 | 0 | 3 |
| chloroquine | 0 | 0 | 4 |

control: 35% Red Blood Cell Infection Rate

### Example 12

### In vivo Efficacy of Atrazine against P. berghei

Rats weighing 60-70 gms were inoculated with P. berghei infected rat red blood cells. Four hours later the rats were given a single dose Chloroquine (20 mg/kg), Atrazine (100 mg/kg) or ethanol (0.1 ml/rat) orally via a gastric gavage tube. Blood was obtained 4 days and 11 days later and parasitemia assessed using light microscopy of blood smears. The percent parasitemia (i.e., the presence of parasites in the blood) was then determined for each group and the data are summarized in the table below:

TABLE 3
Parasitemia 4 and 11 Days After Treatment with Ethanol,
Chloroquine or Atrazine.

| Treatment group | Parasitemia Day 4 | Parasitemia Day 11 |
|---|---|---|
| Ethanol control | 24% | 42% |
| Chloroquine | 5.8% | 11% |
| Atrazine | 6.0% | 10.5% |

### Example 13

### Drug Interactions between Chloroquine and Atrazine.

The drug interactions between Chloroquine and Atrazine were determined using the Isobologram analysis of in vitro cytotoxicity of the two compounds. Drugs can interact in additive, antagonistic or synergistic manner. See Berenbaum, J. Infect. Dis., 137:122-130, 1978. The isobologram assay permits determination of the type of interaction that occurs between drugs of interest. The ID₅₀, or median infectious dose, for both drugs was calculated.

Next, the drugs were tested in vitro at the ID₅₀ for each drug alone and various combinations of drugs as described below:

ID₅₀ Atrazine alone

0.9×ID₅₀ Atrazine+0.1×ED₅₀ Chloroquine

0.75×ID₅₀ Atrazine+0.25×ID₅₀ Chloroquine

0.5×ID₅₀ Atrazine+0.5×ID₅₀ Chloroquine

0.25×ID₅₀ Atrazine+0.75×ID₅₀ Chloroquine

0.1×ID₅₀ Atrazine+0.9×ID₅₀ Chloroquine

ID₅₀ Chloroquine alone.

These combinations of drugs were tested in vitro against P. falciparum and the data analyzed. The results as shown below in Table 4 indicate a clear synergy between the two drugs as regards the inhibition of P. falicarum growth.

TABLE 4
Percent Growth Inhibition of P. falciparum Following Treatment
with Antimalarial Compositions..

| Treatment | Percent Growth Inhibition |
|---|---|
| Atrazine | 58 |
| 90% Atrazine + 10% Chloroquine | 69 |
| 75% Atrazine + 25% Chloroquine | 79 |
| 50% Atrazine + 50% Chloroquine | 87 |
| 25% Atrazine + 75% Chloroquine | 71 |
| 10% Atrazine + 90% Chloroquine | 89 |
| Chloroquine | 60 |

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method for treating humans and animals infected with a parasite of the phylum Apicomplexa, wherein the method comprises administering a therapeutically effective amount of an s-triazine compound.

2. A method for treating humans and animals infected with a parasite of the phylum Apicomplexa, wherein the method comprises administering a therapeutically effective amount of at least one compound with the following formula: 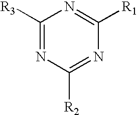
wherein:
R₁, R₂, and R₃ are, independently, hydrogen, halogen, an optionally substituted, linear or branched C₁-C₂₀ alkyl group, an optionally substituted, linear or branched C₂-C₂₀ alkenyl group, an optionally substituted, linear or branched C₂-C₂₀ alkynyl group, an optionally substituted C₃-C₁₂ cycloalkyl group, an optionally substituted C₆-C₂₀ aryl group, an optionally substituted C₃-C₁₂ heterocyclic group containing at least one heteroatom of N, O, or S, OR₄, SR₄, NO₂, NR₄R₅, N═CHR₄, NR₄C(O)R₄, C(O)R₄, C(O)OR₄, or CN, with the proviso that R₁, R₂, and R₃ are not all hydrogen or not all CN groups; and
R₄ and R₅ are, independently, hydrogen, an optionally substituted, linear or branched C₁-C₂₀ alkyl group, an optionally substituted, linear or branched C₂-C₂₀ alkenyl group, an optionally substituted, linear or branched C₂-C₂₀ alkynyl group, an optionally substituted, C₃-C₁₂ cycloalkyl group, an optionally substituted C₆-C₂₀ aryl group, an optionally substituted C₃-C₁₂ heterocyclic group containing at least one heteroatom of N, O, or S, CN, or R₄ and R₅ when taken together with N forms a heterocyclic group;
or a pharmaceutically acceptable salt thereof.

3. The method of claim 2, wherein R₁ is a halogen and R₂ and R₃ are, independently, a NHR₄ group wherein R₄ is a substituted or unsubstituted, linear or branched C₁-C₅ alkyl group.

4. The method of claim 3, wherein R₂ is NHCH₂CH₃, and R₃ is NHCH(CH₃)₂.

5. The method of claim 3, wherein R₂ and R₃ are each NHCH(CH₃)₂.

6. The method of claim 3, wherein R₂ and R₃ are each NHCH₂CH₃.

7. A method for treating humans and animals infected with a parasite of the phylum Apicomplexa, wherein the method comprises administering a therapeutically effective amount of a compound with the following formula: 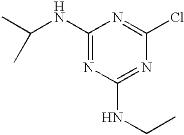
or a pharmaceutically acceptable salt thereof.

8. A method for treating humans and animals infected with a parasite of the phylum Apicomplexa, wherein the method comprises administering a therapeutically effective amount of a compound with the following formula: 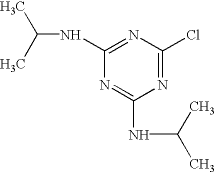
or a pharmaceutically acceptable salt thereof.

9. A method for treating humans and animals infected with a parasite of the phylum Apicomplexa, wherein the method comprises administering a therapeutically effective amount of a compound with the following formula: 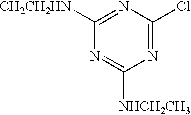
or a pharmaceutically acceptable salt thereof.

10. A method for treating humans and animals infected with a parasite of the phylum Apicomplexa, wherein the method comprises administering a therapeutically effective amount of atrazine.

11. A method for treating humans and animals infected with a parasite of the phylum Apicomplexa, wherein the method comprises administering a therapeutically effective amount of propazine.

12. A method for treating humans and animals infected with a parasite of the phylum Apicomplexa, wherein the method comprises administering a therapeutically effective amount of simazine.

13. A method for treating humans and animals infected with a parasite of the phylum Apicomplexa, wherein the method comprises administering a therapeutically effective amount of 2-chloro-4-ethylamino-6-isopropylamino-s-triazine.

14. A method for treating humans and animals infected with a parasite of the phylum Apicomplexa, wherein the method comprises administering a therapeutically effective amount of 2-chloro-4,6-di(isopropylamino)-s-triazine.

15. A method for treating humans and animals infected with a parasite of the phylum Apicomplexa, wherein the method comprises administering a therapeutically effective amount of 2-chloro-4,6-di(ethylamino)-s-triazine.

16. The method of claims 1-15, wherein the parasite is selected from the group consisting of Plasmodium sp, Toxoplasma sp, Neospora sp, Cryptosporidium sp, Hematodinium sp, Hemogregarines sp, Babesia sp, Eimeria sp, and Theileria sp.

17. The method of claims 1-15, wherein the parasite is of Plasmodium falciparum.

18. A pharmaceutical composition for use in the treatment or prevention of mammalian infection by a parasite of the phylum Apicomplexa, wherein the pharmaceutical composition comprises an anti-microbially effective amount of at least one compound with the following formula: 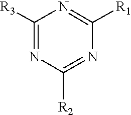
wherein:
R₁, R₂, and R₃ are, independently, hydrogen, halogen, an optionally substituted, linear or branched C₁-C₂₀ alkyl group, an optionally substituted, linear or branched C₂-C₂₀ alkenyl group, an optionally substituted, linear or branched C₂-C₂₀ alkynyl group, an optionally substituted C₃-C₁₂ cycloalkyl group, an optionally substituted C₆-C₂₀ aryl group, an optionally substituted C₃-C₁₂ heterocyclic group containing at least one heteroatom of N, O, or S, OR₄, SR₄, NO₂, NR₄R₅, N═CHR₄, NR₄C(O)R₄, C(O)R₄, C(O)OR₄, or CN, with the proviso that R₁, R₂, and R₃ are not all hydrogen or not all CN groups; and
R₄ and R, are, independently, hydrogen, an optionally substituted, linear or branched C₁-C₂₀ alkyl group, an optionally substituted, linear or branched C₂-C₂₀ alkenyl group, an optionally substituted, linear or branched C₂-C₂₀ alkynyl group, an optionally substituted, C₃-C₁₂ cycloalkyl group, an optionally substituted C₆-C₂₀ aryl group, an optionally substituted C₃-C₁₂ heterocyclic group containing at least one heteroatom of N, O, or S, CN, or R₄ and R₅ when taken together with N forms a heterocyclic group;
or a pharmaceutically acceptable salt thereof.

19. The pharmaceutical composition of claim 18, wherein R₁ is a halogen and R₂ and R₃ are, independently, a NHR₄ group wherein R₄ is a substituted or unsubstituted, linear or branched C₁-C₅ alkyl group.

20. The pharmaceutical composition claim 19, wherein R₂ is NHCH₂CH₃, and R₃ is NHCH(CH₃)₂.

21. The pharmaceutical composition claim 19, wherein R₂ and R₃ are each NHCH(CH₃)₂.

22. The pharmaceutical composition claim 19, wherein R₂ and R₃ are each NHCH₂CH₃.

23. A pharmaceutical composition for use in the treatment or prevention of mammalian infection by a parasite of the phylum Apicomplexa, wherein the pharmaceutical composition comprises an anti-microbially effective amount of a compound with the following formula: 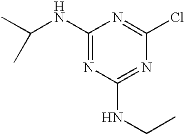
or a pharmaceutically acceptable salt thereof.

24. A pharmaceutical composition for use in the treatment or prevention of mammalian infection by a parasite of the phylum Apicomplexa, wherein the pharmaceutical composition comprises an anti-microbially effective amount of a compound with the following formula: 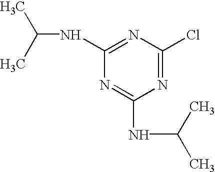
or a pharmaceutically acceptable salt thereof.

25. A pharmaceutical composition for use in the treatment or prevention of mammalian infection by a parasite of the phylum Apicomplexa, wherein the pharmaceutical composition comprises an anti-microbially effective amount of a compound with the following formula: 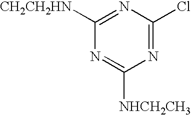
or a pharmaceutically acceptable salt thereof.

26. A pharmaceutical composition for use in the treatment or prevention of mammalian infection by a parasite of the phylum Apicomplexa, wherein the pharmaceutical composition comprises an anti-microbially effective amount of an s-triazine compound.

27. A pharmaceutical composition for use in the treatment or prevention of mammalian infection by a parasite of the phylum Apicomplexa, wherein the pharmaceutical composition comprises an anti-microbially effective amount of atrazine.

28. A pharmaceutical composition for use in the treatment or prevention of mammalian infection by a parasite of the phylum Apicomplexa, wherein the pharmaceutical composition comprises an anti-microbially effective amount of propazine.

29. A pharmaceutical composition for use in the treatment or prevention of mammalian infection by a parasite of the phylum Apicomplexa, wherein the pharmaceutical composition comprises an anti-microbially effective amount of simazine.

30. A pharmaceutical composition for use in the treatment or prevention of mammalian infection by a parasite of the phylum Apicomplexa, wherein the pharmaceutical composition comprises an anti-microbially effective amount of 2-chloro-4-ethylamino-6-isopropylamino-s-triazine.

31. A pharmaceutical composition for use in the treatment or prevention of mammalian infection by a parasite of the phylum Apicomplexa, wherein the pharmaceutical composition comprises an anti-microbially effective amount of 2-chloro-4,6-di(isopropylamino)-s-triazine.

32. A pharmaceutical composition for use in the treatment or prevention of mammalian infection by a parasite of the phylum Apicomplexa, wherein the pharmaceutical composition comprises an anti-microbially effective amount of 2-chloro-4,6-di(ethylamino)-s-triazine.

33. The pharmaceutical composition of claims 18-32 further comprising one or more other anti-parasitic compounds.

34. The pharmaceutical composition of claim 33, wherein the additional anti-parasitic compounds are selected from the group consisting of proguanil, chloroquine, pyrimethamine, mefloquine and quinine.

35. The pharmaceutical composition of claims 18-32 further comprising one or more pharmaceutically acceptable carriers.
